(19) United States
(12) Patent Application Publication
(10) Pub. No.: US 2003/0083559 A1
(43) Pub. Date: May 1, 2003

(54) Non-contact monitor

(76) Inventor: David L. Thompson, Andover, MN (US)

Correspondence Address: MEDTRONIC, INC., 710 MEDTRONIC PARKWAY NE, MS-LC340, MINNEAPOLIS, MN, 55432-5604, US

(21) Appl. No.: 09/999,827

(22) Filed: Oct. 31, 2001

(51) Int. Cl. A61B 5/04

(52) U.S. Cl. 600/372

(57) ABSTRACT

A peripheral monitor patch apparatus for attachment to a patient includes a high capacity memory for the storing and later retrieving of the sensed and compressed physiologic data sensed by unique electrodes. A resilient substrate provides support for a memory, microprocessor, receiver, and other electronic components. The substrate flexes in a complimentary manner in response to a patient's body movements. The substrate is affixed to the patient's skin or clothes with the use of an adhesive, which provides for comfort and wearability. The low profile peripheral patch apparatus is preferably similar in size and shape to a standard bandage, and may be attached to the patient in an inconspicuous location. A status indicator provides for a visual, verbal, or tactile indication of the operational status of the peripheral monitor patch.

### FIELD OF THE INVENTION

This invention relates to a non-implantable monitoring device for sensing and storing physiologic events with no intrusion into a human/patient body, and is particularly well suited for long term monitoring of body events like electrocardiograms (ECG's) and in detecting/monitoring other body physiologic events related to physiologic function which may include, for example, without limitation, such parameters as arrhythmia detection, syncope, QT variability, ischemia, and respiration relevant to normal and abnormal physiologic function. By enabling easy monitoring and recording of physiologic events in the patient's body, such events can then be studied at leisure outside the body, providing diagnostic, therapeutic and research opportunities not otherwise available.

### BACKGROUND OF THE INVENTION

Syncopal events and arrhythmias of the heart are particularly problematic for diagnostic physicians to observe in living patients. These events, can be of short duration and sudden onset, coming with little or no warning, and may occur very infrequently. Holter monitors are well known for monitoring electrocardiograms for repetitive 24-hour periods of time often amounting to several days or perhaps a week, but these are expensive, bulky, applied externally to the body and interfere with the patient's normal life, making them impractical for long term use. Further, patient compliance cannot always be guaranteed, and is a common problem in use of the Holter devices. Problems with external monitors and associated recorders also include inability of some patients to tolerate the attendant skin irritation from the long-term electrode attachment.

Bulky or expensive special purpose recording devices may need to be available and maintained. Removal (recorder and electrodes) is required for showering, and so on. Any time a living body needs to have a long term monitoring of a physiologic event that is intermittent or infrequent or both, all these problems come into focus. Therefore, there exists a need for minimally intrusive long-term monitoring of the patient's physiologic events and medical status. This is particularly indicated in, but not limited to patients with cardiac arrhythmias and vasovagal syncope to provide sufficient evidence for diagnostic purposes, subsequent therapy/treatment, and for research into the causes and effects of such events.

The problem has long existed and many attempts to address some of these problems have been made and met with limited success. Examples of an external monitor/recorders (ie, Holter monitor) can be found in Segalowitz's patents, including U.S. Pat. Nos. 4,981,141; 5,168,874; 5,307,818 and 5,511,553; Salo's U.S. Pat. No. 5,417,717; Platt's U.S. Pat. No. 5,634,468; Dougherty's U.S. Pat. No. 5,027,824; and Suzuki's U.S. Pat. Nos. 5,007,427 and 5,111,818. All require multiple tissue-contacting, adhesively-attached electrode systems and skin preparation (i.e., shaving, “sanding” of the skin) and application of a conductive gel are required for good electrical electrode contact.

Alternatively, a wrist-worn monitor for ECG's that include features like patient triggering and microprocessor determination of event types (QRS detection) is seen in the Mills, et al patents (U.S. Pat. Nos. 5,333,616; 5,289,824 and 5,111,396). Wrist-worn devices are also shown in the Righter patents, including U.S. Pat. Nos. 5,226,425 and 5,365,935. Motion artifacts during patient movement and the removal for showering/bathing are known problems with this technique.

Implantable monitors have also been proposed as a solution to the problem. For example, U.S. Pat. No. 5,987,352 to Klein, et al describes a minimally invasive implantable device with preferably a segmented looping memory for storing triggered physiologic events which may include arrhythinias and syncopal events. A patient activated manual trigger is included. Automatic triggers and manually set triggers may be of different sizes. Preferred communications with the device is through telemetry such as is used for pacemakers and other implanted devices.

Yomatov's patents, U.S. Pat. Nos. 5,411,031 and 5,313,953 describe an implantable cardiac monitor arranged for detecting both arrhythmias and ischemia of the human heart. The monitor includes subcutaneous electrodes for establishing electrical contact with the heart and a sense amplifier coupled to each electrode for generating an electrocardiogram of a heart beat sensed at each of the electrodes. The electrocardiograms are digitized and the digital samples thereof are stored in a memory. A microprocessor processes the digital samples of the electrocardiograms and generates characterizing data indicative of the physiology of the heart. The cardiac monitor includes telemetry to permit the cardiac data to be interrogated externally of the patient for obtaining the generated cardiac data indicative of arrhythmic and ischemic episodes.

Monitoring can also be done using implantable medical devices (IMDs) such as pacemakers and other heart stimulating devices or devices with leads in the heart for capturing various physiologic parameters, including the intracardiac electrogram (ECG). Such devices, typically with leads fixed in the patient's heart, in addition to performing therapeutic operations, may monitor and transmit cardiac electrical signals (ECG) to an external diagnostic device. It is common for implanted cardiac stimulation devices to send ECG signals to a monitoring device, such as an external programmer, to allow a user to observe and analyze the interaction between the heart and the implanted device. Often the user can designate that the communication from the implantable device to the programmer include a transmission of codes which signal the occurrence of a cardiac event such as the delivery of a stimulation pulse or a sensed spontaneous cardiac depolarization such as substantially described in U.S. Pat. No. 4,374,382 to Markowitz.

Exemplary IMD devices include, for example, U.S. Pat. No. 4,223,678, entitled “Arrhythmia Recorder for Use with an Implantable Defibrillator”, issued to Langer et al., which discloses an arrhythmia record/playback component within an implantable defibrillator. ECG data is converted from analog to digital (A/D) form and stored in a first-in, first-out looping memory. When the defibrillator detects an arrhythmia event, it disables the memory so that no further ECG data is recorded in the memory until a command is received from an external monitoring device. This command requests the implantable defibrillator to transmit the stored ECG data to the monitoring device via telemetry. Langer et al. in U.S. Pat. No. 4,407,288, entitled “Implantable Heart Stimulator and Stimulation Method”, discloses a programmable, microprocessor based implantable defibrillator which senses and loads ECG data into a memory via a direct memory access operation. A processor analyzes this ECG data in the memory to detect the occurrence of an arrhythmia event afflicting a patient's heart. Upon such an event, the defibrillator may generate a therapy to terminate the arrhythmia event and store the ECG data sequence of the event, for transmission to an external monitoring device and later study. In normal circumstances, when no arrhythmia event is occurring, the defibrillator continuously overwrites the ECG data in the memory.

U.S. Pat. No. 4,556,063, entitled “Telemetry System for a Medical Device”, granted to Thompson et al, 1985, teaches a pulse interval telemetry system capable of transmitting analog data, such as sensed intracardiac electrogram signals, without converting analog data to a digital numeric value. The Thompson et al. telemetry system is capable of sequentially transmitting both digital and analog data, individually and serially, in either an analog or a digital format, to a remote receiver. The features and capabilities of these pacemaker/defibrillator devices are now well known, but the problems in long term monitoring for events and adequate recordation remain.

With sufficient additional hardware and connections to the body, numerous other physiologic parameters may be sensed as is pointed out in U.S. Pat. No. 5,464,434 issued to Alt describes a medical interventional device that is adapted to be implanted in a patient's body to provide a number of different controllable therapeutic functions including cardiac pacing, antitachycardia pacing, cardioversion and defibrillation. A sensor generates an electrical signal representing sensed variations of a physiologic parameter of the patient indicative of the substantially instantaneous hemodynamic condition of the patient. The physiologic parameter sensed may be any of blood pressure, blood oxygen content, minute ventilation, central venous temperature, pulse rate, blood flow, physical activity, or other parameter for that purpose. A computer calculates the mean and standard deviation of the generated signal over a predetermined time interval, and especially the quotient of the standard deviation and the mean from which to determine a sudden hemodynamic change such as a precipitous drop in cardiac output. The device accepts this as an indication of syncope warranting intervention with a defibrillating protocol, and triggers such response.

However, the expense and risk from implanting intracardiac leads, and/or sensors, and associated IMD such as a pacemaker or defibrillator with special monitoring functions for short-term diagnostic or monitoring use is something both patients and physicians would prefer to avoid.

Accordingly, there still exists a need for an inexpensive, non-invasive, more acceptable recording and monitoring device capable of maintaining a data record over a long period of time and highlighting or least capturing those physiologic events that are of interest to a diagnostic, research or therapeutic study, and particularly those physiologic events that are required for the correct diagnosis and therapy of effected patients. Further, it has heretofore been unreasonably expensive and overly invasive to the patient to implant monitors for simple recording functions and particularly to implant intracardiac and intravascular monitors for simple recording functions. Lastly, external monitors are expensive, bulky, and the skin preparation and the long term attachment of EKG electrodes are also problematic. Many of the features of this invention are designed to ameliorate all of these problems.

### SUMMARY OF THE INVENTION

The present invention includes a non-tissue contacting electrode system for the sensing of physiologic signals from a patient that may be used during the diagnostic monitoring of patients with various cardiac arrhythmias or anomalies. These sensing systems may transmit the stored data to the circuitry of an external display device and allow the detection of cardiac arrhythmias and/or other physiologic anomalies.

The present invention provides a method and apparatus that may be implemented to provide an enhanced capability of detecting and gathering electrical cardiac signals via non-tissue contacting sensors.

The present invention allows the physician or medical technician to perform follow-up that, in turn, eliminates the time it takes to attach external adhesive electrodes to the patient's skin and provide for a more robust long-term signal acquisition. Such time-savings can reduce the cost of follow-up, as well as making it possible for the physician or medical technician to see more patients during each day. Additionally, the present invention is easier for the patient to utilize for extended periods of time. Though not limited to these, other uses include: cardiac monitoring with event storage, arrhythmia detection and monitoring, capture detection, ischemia detection and monitoring (S-T elevation and depression on the ECG), changes in QT interval (i.e., QT variability), and transtelephonic monitoring.

Specific aspects of the present invention include providing a minimally intrusive system capable of communicating with a monitoring service and having special electrodes to sense and measure an electrogram including a signal input means, here shown as an amplifier, a looping memory, and a circuit for controlling the memory, the device having an external configuration and dimensions maximally adapted to such needs.

A preferred data compression scheme is also disclosed as is automatic selection of time periods pre and post triggering.

In its presently most preferred embodiment the invention provides for long term ECG monitoring and includes capacity to use manual or automatic triggers or both to cause the memory to store events in reserved areas of a looping memory, preferably in identifiable memory partitions. The non-contact monitor of the present invention can accept programming or mode control and can read out sections of or all of the memory when prompted from the outside by a physician or other user, provided the user has the appropriate external device to initiate and receive such transmissions from the non-contact monitor.

### BRIEF DESCRIPTION OF THE DRAWINGS

FIG. 1 shows a peripheral physiologic signal recorder attached to a human body that detects and stores physiologic, diagnostic, and other information in accordance with an embodiment of the present invention;

FIG. 2 shows various electrical, electronic, and structural elements of a peripheral monitor patch from a top view perspective in accordance with an embodiment of the present invention;

FIG. 3 shows the peripheral monitor patch of FIG. 2 from a cross-sectional view perspective;

FIG. 4 is a block diagram illustrating the main circuit and assembly of a device in accord with a preferred embodiment;

FIGS. 5A-D are block diagrams of preferred embodiment circuits of the implanted device used for monitoring and storing ECGs;

FIG. 6 is a flow chart of the functioning of the recordation of triggered events in a preferred embodiment of the invention;

FIG. 7 is a display of an ECG tracing from a capacitive, non-tissue contacting sensor in accordance with one embodiment of the invention;

FIG. 8 is a display of an ECG tracing from a non-acoustic, pulse-echo radar, non-tissue contacting sensor in accordance with an alternative embodiment of the invention; and

FIG. 9 is a flow diagram of a method of using the apparatus of the present invention.

FIG. 10 represents a remote communication and data management system incorporated with the present invention.

### DETAILED DESCRIPTION OF THE PREFERRED EMBODIMENT

In the following description of the illustrated embodiments, references are made to the accompanying drawings that form a part hereof, and in which is shown by way of illustration, various embodiments in which the invention may be practiced. A variety of embodiments of a peripheral monitor patch attached on a patient's skin so as to define various system embodiments of the present invention that provide for the recording of physiologic, diagnostic, and other information may be recorded. It is to be understood that other embodiments may be utilized, and structural and functional changes may be made without departing from the scope of the present invention.

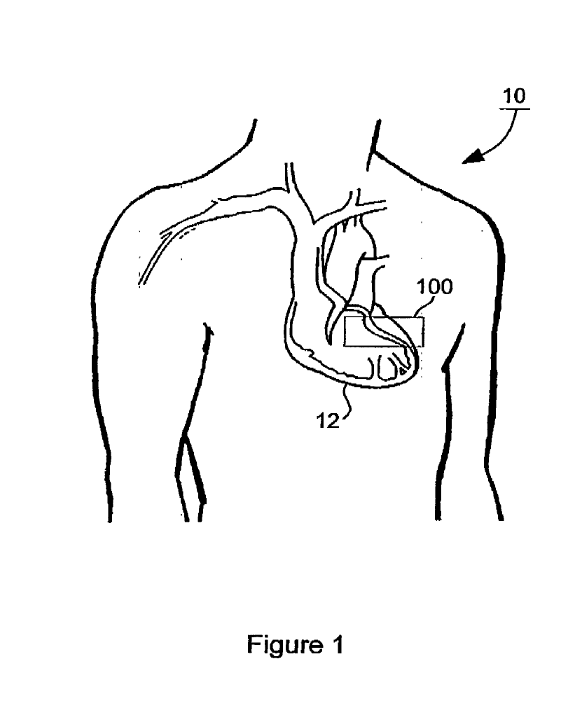

FIG. 1 is a simplified schematic view of a peripheral monitor patch 100 of the present invention shown affixed to the skin of a human body 10 over, or the vicinity of, the patient's heart 12.

In general, the peripheral monitor device 100 shown in FIG. 1 includes a flexible tape structure with an enclosure that may include various elements, such as an electrochemical cell (e.g., a lithium battery), circuitry that controls device operation and records arrhythmic EGM episodes, and an optional communication circuit for the transmission of stored data to an external system and/or display.

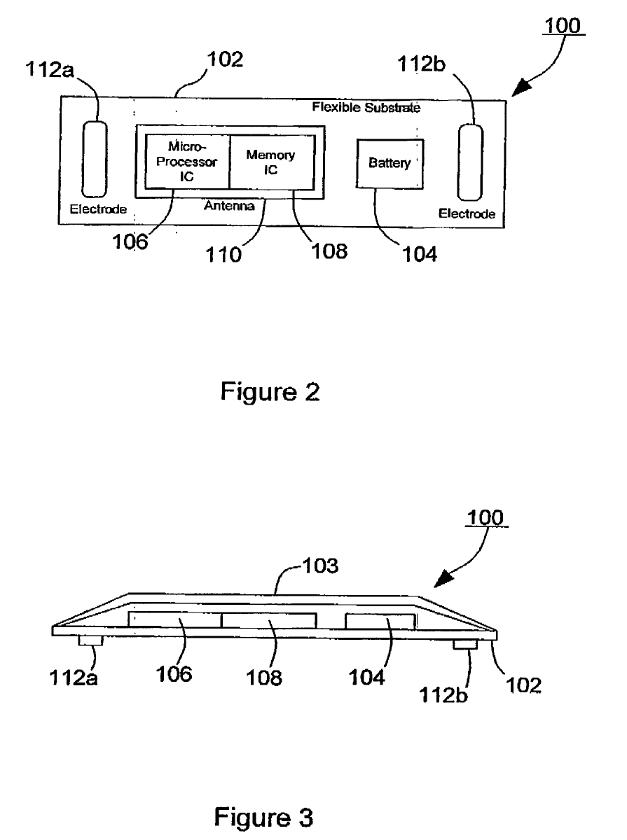

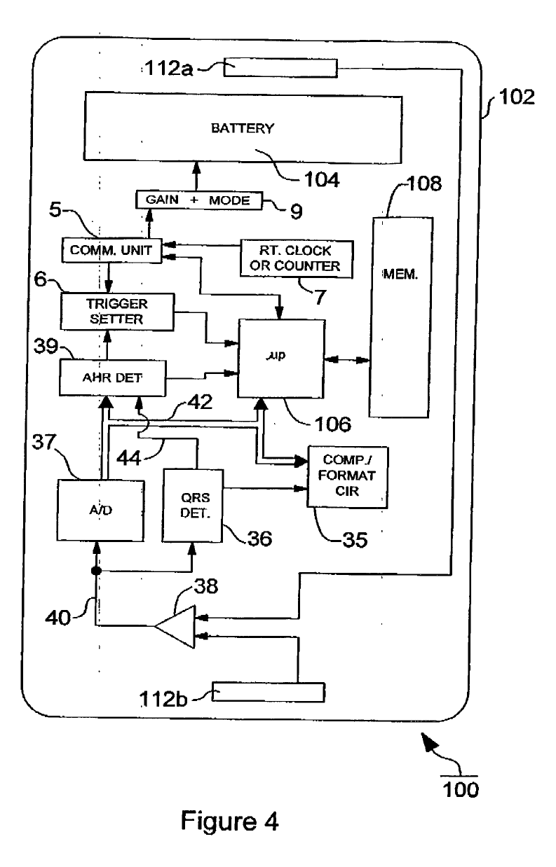

FIGS. 2 and 3 respectively show top and side cross-sectional views of a peripheral monitor module in accordance with an embodiment of the present invention. A peripheral monitor patch 100, in accordance with the embodiment depicted in FIG. 4, is provided on a flexible substrate 102. Substrate 102 includes an adhesive backing (not shown) that provides for both comfort and extended periods of wear when affixed directly on the patient's skin or clothing.

It is contemplated that peripheral monitor patch 100 may be attached to a patient's skin for periods of about one to two days, or longer. Although extended periods of wear on the order of one to two weeks may be desirable or necessary under certain circumstances, it is known that such long-term continuous contact between the adhesive and skin can result in minor itching problems. Additionally, good electrode contact over extended periods of time are also problematic. In an alternative embodiment, substrate 102 may be flexible or rigid, and may be provided with a hook and loop type of securing arrangement to affix peripheral monitor patch 100 to a piece of clothing worn by the patient.

In a first embodiment, flexible substrate 102 may include hydrophilic pressure sensitive adhesives and, in addition, may have a construction similar to that disclosed in U.S. Pat. Nos. 5,489,624 and 5,536,768, both of which are hereby incorporated by reference herein in their respective entireties. Flexible substrate 102 may have a size and shape similar to that of commercially available disposable bandages. In one embodiment, flexible substrate 1402 has a width dimension ranging between approximately 0.5″ and approximately 3″, and a length dimension ranging between approximately ¾″ and approximately 5″. Peripheral monitor patch 100 may have a thickness dimension ranging between approximately 0.025″ and approximately 0.25″.

In the embodiment illustrated in FIGS. 2 and 3, flexible substrate 102 may comprise a resilient material upon which several electronic and electrical components are mounted. Flexible substrate 102 may include an integral or separate interconnect pattern of electrical conductors that provide for interconnection between the various components disposed on flexible substrate 102. Suitable materials that may be used to fabricated flexible substrate 102 include Mylar, flexible foil, flex PC, Kapton, and polymer thick film (PTF).

The electronic portion of peripheral monitor patch 100 includes a microprocessor integrated circuit (IC) 106 and a memory IC 108. Shown surrounding microprocessor 106 and memory 108 is an optional antenna 110 that transmits physiologic data to a remote data collection or monitoring systems. Also provided on flexible substrate 102 is a battery 104 and one or more sensors 112a and 112b. In one embodiment, flexible substrate 102 and the various components populating flexible substrate 102 which define the electronics module of peripheral monitor patch 100 are fabricated and packaged in accordance with various known “smart card” technologies, examples of which are disclosed in U.S. Pat. Nos. 5,311,396 and 5,480,842, both of which are incorporated herein by reference in their respective entireties.

The electronics module of peripheral monitor patch 100 may include a flexible foil substrate 102 with an attached battery 104 and chip-on-board (COB) memory chips 108. In accordance with one embodiment, battery 104 may have a lithium manganese oxide (e.g., LiMnO2) chemistry, and may be of a sealed foil design. Although a rectangular shape to the various components is shown in FIGS. 2 and 3, various other component geometries, such as square, round or oval shapes, may be employed.

Memory 108 may constitute a single memory IC or several memory ICs. Memory 108 is preferably a state-of-the-art, commercially-available memory that may be embodied in various memory technologies (e.g., CMOS). Memory 108, for example, may include one or more dynamic random access memories (DRAMs), static random access memories (SRAMs), electrically erasable programmable readonly memories (EEPROMs), flash memories, ferroelectric memories, and/or analog memories.

Refer now to FIG. 4 in which a peripheral monitor 100 is illustrated in an outline of the peripheral monitor flexible substrate 102. Sensors 112a and 112b bring the signal from the body to an input differential amplifier 38 for simplicity only, the output of which is fed to a QRS detector 36 and an A/D converter 37. Both circuits, detector 36 and A/D converter 37, supply outputs to an arrhythmia detector 39, which in this preferred embodiment supplies the autotrigger signal to the trigger setting circuit 6. The data output from the Analog to Digital Converter 37 (A/D) may be converted, compressed, formatted and marked or reformulated if desired in a compression/formatting circuit 35 before the data is ready for input into the memory 108. The Memory control circuit 8 receives input from the A/D converter 37, from the arrhythmia detection circuit 39 (which may include input directly from the QRS detector if desired) as well as signals from the trigger setter circuit 6. The trigger setter circuit 6 may also be controlled by a communications unit 5 that operates to communicate to an outside device (not shown) by receiving/transmitting and decoding/encoding signals that are telemetered or otherwise communicated to a user. This communications unit 5 will also be able to communicate with the memory controller to request the offloading of memory data for analysis by an outside interrogation device. It should contain an antenna and transceiver circuitry, or alternatively, IrDA circuitry to communicate with an outside device. A clock circuit 7 reports the time since the device was started or real time to the outside interrogator device contemporaneously with a data offloading session so that the events recorded in memory 108 may be temporally pinpointed.

Alternatives to this overall design may be considered, for example by using a custom digital ASIC to accomplish some or all of the functions of microprocessor circuit 106.

Electrodes 112a and b of FIGS. 2 and 3 are positioned on the surface of flexible substrate 102 facing the patient. Electrodes 112a and 112b may be as substantially described in PCT application WO 01/16607, Electric Field Sensor, by Brun del Re, et al, incorporated by reference in its entirety. The Brun del Re '607 application describes an electric field sensor employing a capacitive pickup electrode in a voltage divider network connected to a body emanating an electric field. The system is relatively insensitive to variations in the separation gap between electrode and body, reducing sensor motion artifacts in the output signal and stabilizing its low frequency response. The pick-up electrode may be positioned at a “stand off” location, spaced from intimate contact with the surface of the body. Human body-generated electrical signals may be acquired without use of conductive gels and suction-based electrodes, without direct electrical contact to the body and even through layers of clothing.

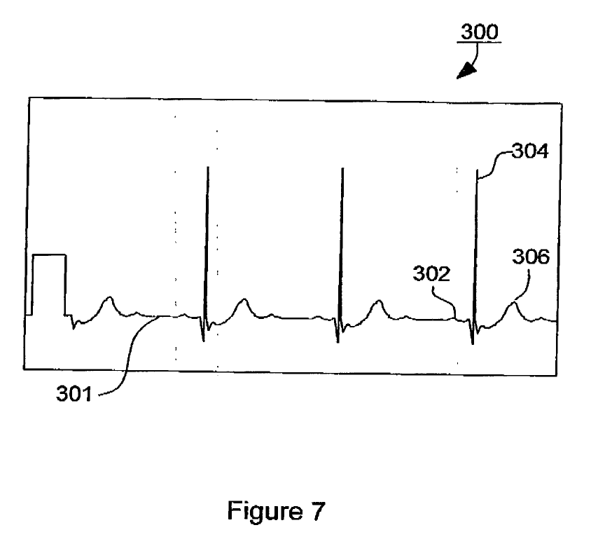

FIG. 7 displays an ECG tracing 301, superimposed upon a grid 300, taken during an acute study from a human patient utilizing the sensors 112a and 112b of FIGS. 2 and 3, showing a p-wave 302, a QRS complex 304, and a T-wave 306. These signals may be applied to the arrhythmia detection circuitry as described in FIGS. 4 above and 5A-D herein below.

In a second embodiment, sensors 112a and 112b may be as substantially described in U.S. Pat. Nos. 5,573,012; 5,966,090; and 5,986,600 by McEwan, incorporated by reference in their entireties. The McEwan '012, '090, and '600 patents describe a non-acoustic pulse-echo radar monitor, employed in a repetitive mode, whereby a large number of reflected pulses are averaged to produce a voltage that corresponds to the heart motion. The antenna used in this monitor generally comprises two flat copper foils, thus permitting the antenna to be housed in a substantially flat housing. It further uses a dual time constant to reduce the effect of gross sensor-to-surface movement. The monitor detects the movement of one or more internal body parts, such as the heart, lungs, arteries, and vocal chords, and includes a pulse generator for simultaneously inputting a sequence of pulses to a transmit path and a gating path. The pulses transmitted along the transmit path drive an impulse generator and provide corresponding transmit pulses that are applied to a transmit antenna. The gating path includes a range delay generator that generates timed gating pulses. The timed gating pulses cause the receive path to selectively conduct pulses reflected from the body parts and received by a receive antenna. The monitor output potential can be separated into a cardiac output indicative of the physical movement of the heart, and a pulmonary output indicative of the physical movement of the lung.

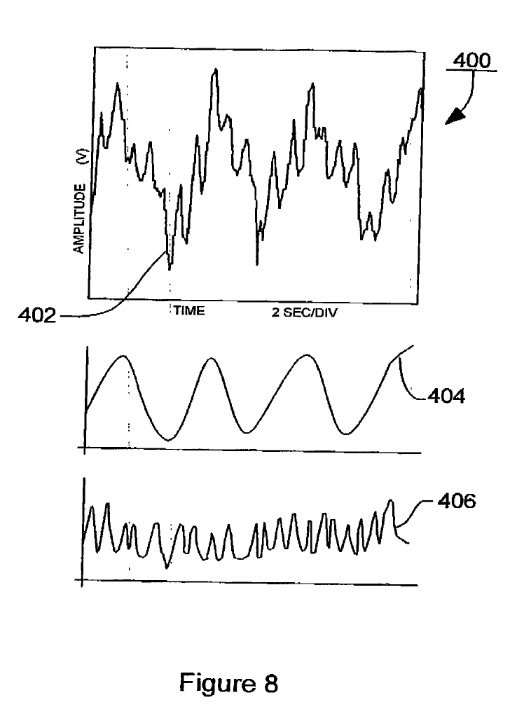

FIG. 8 displays a physiologic waveform 402, superimposed upon a grid 400, taken during an acute study from a human patient utilizing the sensors 112a and 112b of FIGS. 2 and 3. The composite signal 402 is low pass filtered to remove the cardiac signal component with the respiration signal 404 remaining. Respiration signal 404 consists of respiration rate (frequency of breathing) and tidal volume (amplitude). This signal may be used by the follow-up clinician or technician to monitor and/or optimize the performance of respiration-based rate responsive pacemakers, such as substantially described in U.S. Pat. No. 4,919,136, Ventilation Controlled Rate Responsive Cardiac Pacemaker, to Alt, incorporated herein by reference in its entirety. Additionally, this signal may be used to monitor and/or optimize emphysema, edema or CHF patients as described in U.S. Pat. Nos. 5,957,861 Impedance Monitor For Discerning Edema Through Evaluation of Respiratory Rate to Combs, et al, and U.S. Pat. No. 5,876,353 Impedance Monitor For Discerning Edema Through Evaluation of Respiratory Rate to Riff, incorporated herein by reference in their entireties.

The composite signal 402 is high pass filtered to remove the tidal volume signal component with the cardiac signal 406 remaining. The cardiac component may be applied to the arrhythmia detection circuitry as described in FIGS. 4 above and 5A-D herein below.

The cardiac signal 406 may be additionally used to detect and assess acute ischemia via ST segment elevation and depression such as described in U.S. Pat. No. 6,115,628, Method and Apparatus For Filtering Electro-Cardiogram (ECG) Signals to Remove Bad Cycle Information and For Use Of Physiologic Signals Determined From Said Filtered ECG Signals to Stadler, et al; U.S. Pat. No. 6,115,630, Determination of Orientation of Electrocardiogram Signal in Implantable Medical Devices to Stadler, et al; and U.S. Pat. No. 6,128,526, Method for Ischemia Detection and Apparatus for Using Same to Stadler, et al. The Stadler '628, '630, and '526 patents are incorporated herein by reference in their entireties.

The cardiac signal 406 may additionally be used to measure and assess QT variability as described in U.S. Pat. No. 5,560,368, Methodology for Automated QT Variability Measurement to Berger. The Berger '368 patent is herein incorporated by reference in its entirety.

The cardiac signal 406 may additionally be used to remotely monitor and transmit EKG signals from a patient's home without wrist electrodes or tape-on electrodes as described in U.S. Pat. No. 5,467,773, Cardiac Patient Remote Monitoring Using Multiple Tone Frequencies From Central Station to Control Functions of Local Instrument at Patient's Home to Bergelson, et al. The Bergelson '773 patent is incorporated herein by reference in its entirety. These signals may be transmitted via cellular means or, alternatively, by the Internet to a remote monitoring station as described in U.S. Pat. No. 5,752,976, World Wide Patient Location and Data Telemetry System for Implantable Medical Devices to Duffin, et al; U.S. Pat. No. 6,292,698 World Wide Patient Location and Data Telemetry System for Implantable Medical Devices to Duffin, et al; U.S. Pat. No. 6,083,248, World Wide Patient Location and Data Telemetry System for Implantable Medical Devices to Thompson; U.S. patent application Ser. No. 09/348,506, System for Remote Communication With a Medical Device to FerekPetric, filed Jul. 7, 1999; U.S. Provisional application Ser. No. 09/765,484, System and Method of Communicating Between an Implantable Medical Device and a Remote Computer System or Health Care Provider to Haller, et al, filed Jan. 18, 2001; U.S. Pat. No. 5,772,586, Method for Monitoring the Health of a Patient to Heinonen; and U.S. Pat. No. 5,113,869, Implantable Ambulatory Electrocardiogram Monitor to Nappholz. The Duffin '976, '698; Thompson '248; Heinonen '586; and Nappholz '869 patents and Ferek-Petric '506 and Haller '484 applications are herein incorporated by reference in their entireties.

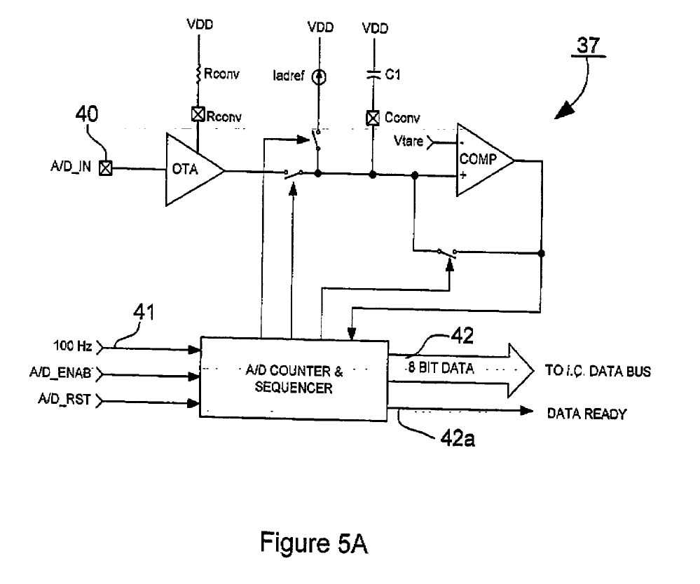

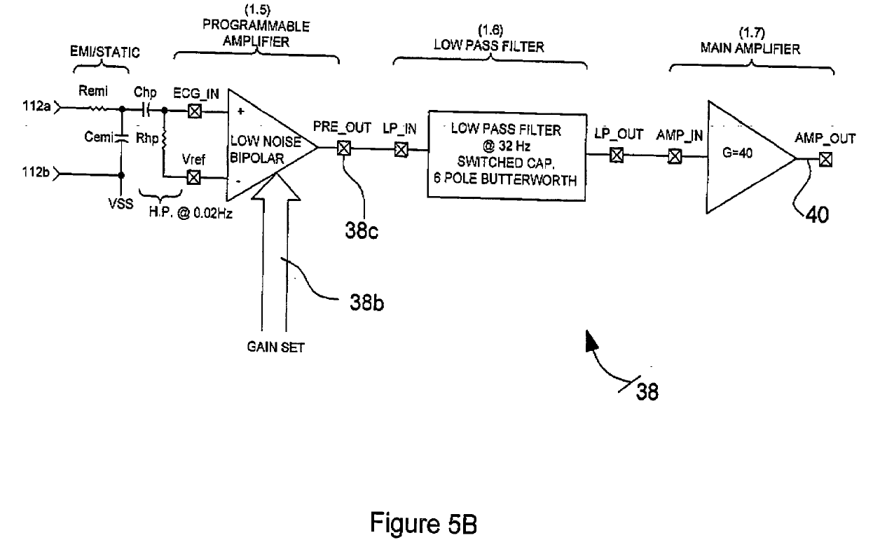

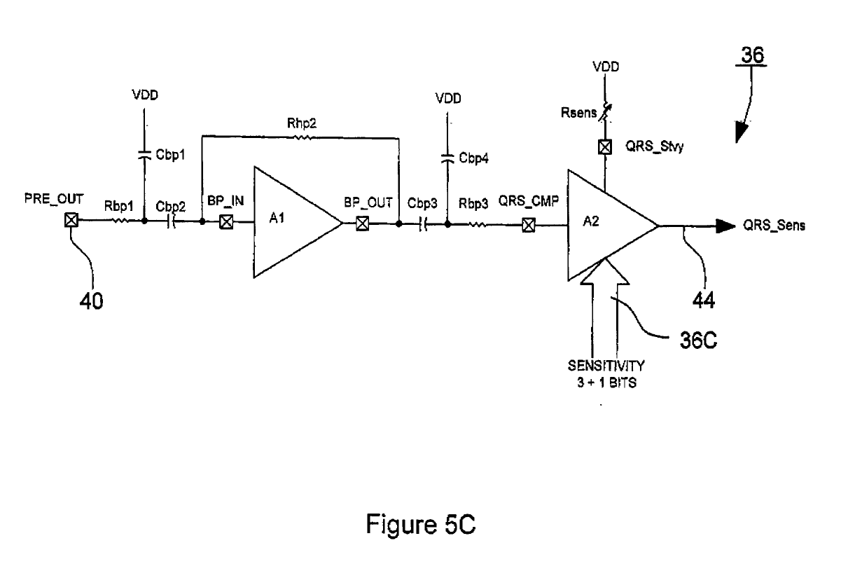

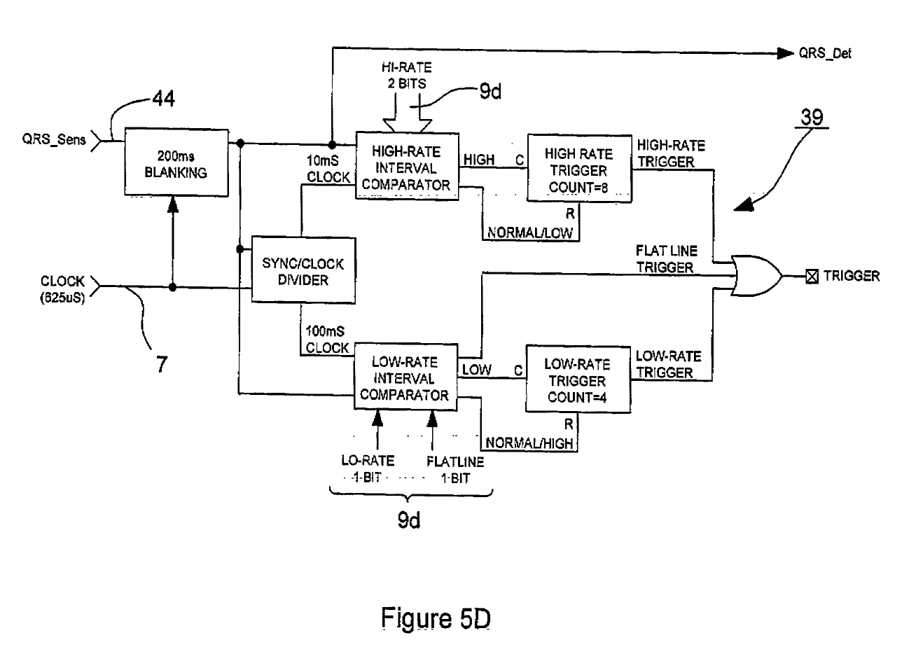

In FIG. 5A, a block diagram of an analog to digital conversion circuit 37 of FIG. 4 for use in this invention is shown. The clock input may advantageously use an output from the clock circuit 7, input 41. The A/D converter input 40 is the analog input signal from input amplifier 38, and the converted output is a stream of 8 bit digital data words on data bus 42, sequenced by timing signal 42a.

FIG. 5B illustrates the basic parts of circuit 38, additionally indicating the input of gain set bits which can modify the value of the output of the low noise amplifier for output at line 38c, the input to the QRS detector. In this invention QRS event detection is done on the analog signal, advantageously saving battery power by only allowing more complex detection after digital conversion has been completed.

In FIG. 5C QRS detect circuit 36 has a 2nd order bandpass filter with a center frequency preferably in the 20-25 Hz range. It includes a transconductance amp A1, summing amp/comparator A2 and resistors Rbp1-3, capacitors Cbp1-4 and selectable resistor R sense connected as shown. R sense is preferably adjusted during manufacture. Additional control is provided for QRS sensitivity at line 36c, since the gain is selectable by this input.

A simple arrhythmia detection circuit 39 is included with this preferred embodiment, and illustrated in FIG. 5D. The output from circuit 36 is monitored at a 200 millisecond blanking interval circuit, controlled by a clock input from clock 7, FIG. 4. In the preferred embodiment, a high rate can be selected amongst 4, with two selection bits dedicated to do so at input 9d and the low and flatline trigger rates each have one bit to turn them on or off provided by inputs 9d. These inputs designated 9d preferably come from a register that holds the gain, the mode and the rate settings, illustrated as register 9 in FIG. 4. Such features may be programmable through communication with the implanted device by an external device. Preferred timing for the high rate triggers is 140, 162 and 182 beats per minute, requiring 8 consecutive beats at such a rate to initiate the trigger. Additionally the trigger may be programmed off. The low rate counter/comparator may be programmable to detect low rates of 40 or 30 BPM, requiring 4 consecutive low rate intervals to trigger. Additionally a flat-line trigger can be set to occur after 3 or 4 and one half seconds of no QRS detection.

For embodiments that include more sensors and/or electronics, additional sensors could be added to benefit the patient. One particularly useful would be an activity sensor based on a single or multi-axis accelerometer, which indicates the level of patient activity and his orientation. By checking for output that indicates the occurrence of a VVS (Vaso Vagal Syncope) episode, (for example, the patient falling from an episode) such an addition offers an improved trigger for events that might otherwise be missed by an arrhythmia detector set up like in FIG. 5D. Such a sensor trigger could replace the circuitry of 5D.

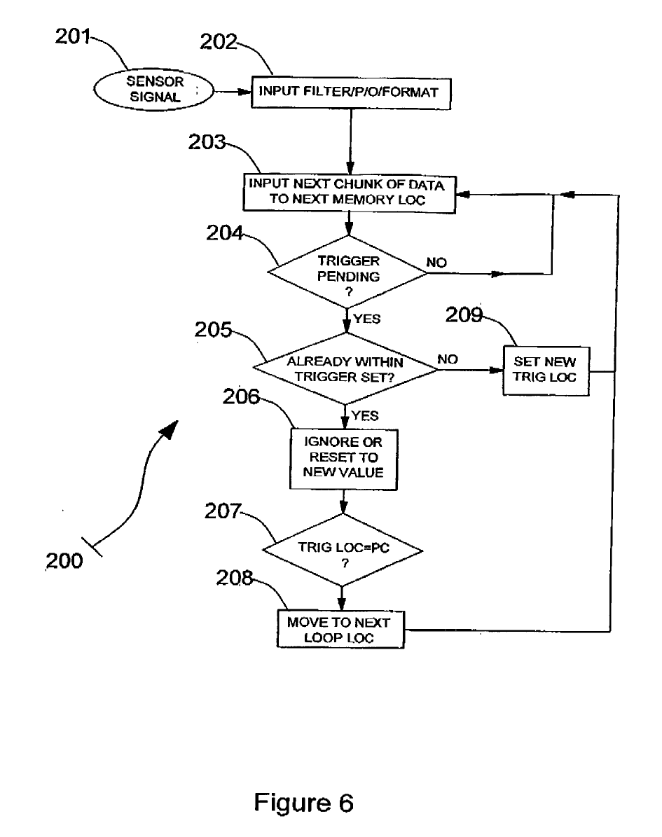

The diagrammatic algorithm 200 to indicate the flow of this information is found in the illustration of FIG. 6 in which a sensor signal 201 is input filtered, converted from analog input to digital values, compressed and formatted if desired in step 202 so as to be in appropriate form to store in a memory location designated by a program counter pointer.

This data word's form could be containing a value representing input signal compressed at various available ratios, and may be mixed with other information like data provided by another sensor or clock data. The data stored will of course carry information related to the signal taken at the sampling rate. Thus lower sampling rates to save power will adversely affect the usefulness or detail of the data. Whatever its preferred form, each data point stored as a word is referred to as a chunk.

Output form step 202 provides the next chunk of data to the next memory location in step 203. The device checks to see if there is any trigger pending after storing each chunk of data in step 204. If not, the next chunk of data is stored. If there is, the device preferably checks to see if there is another trigger already set and, if so, either ignores it or resets the value of the reserved looping memory area (108, FIG. 4) to accommodate a larger trigger or it ignores the trigger if it is smaller or if it indicates a smaller value needs to be stored. If on the other hand, no trigger is already set, then a new trigger location is recorded in the trigger location memory and then the next memory location is written with the next chunk of data. At step 207 if the trigger location is equal in value to the program counter, the device knows that it has gone through the entire loop reserved by the mode selector for this particular event record and then moves on to the next loop location, step 208.

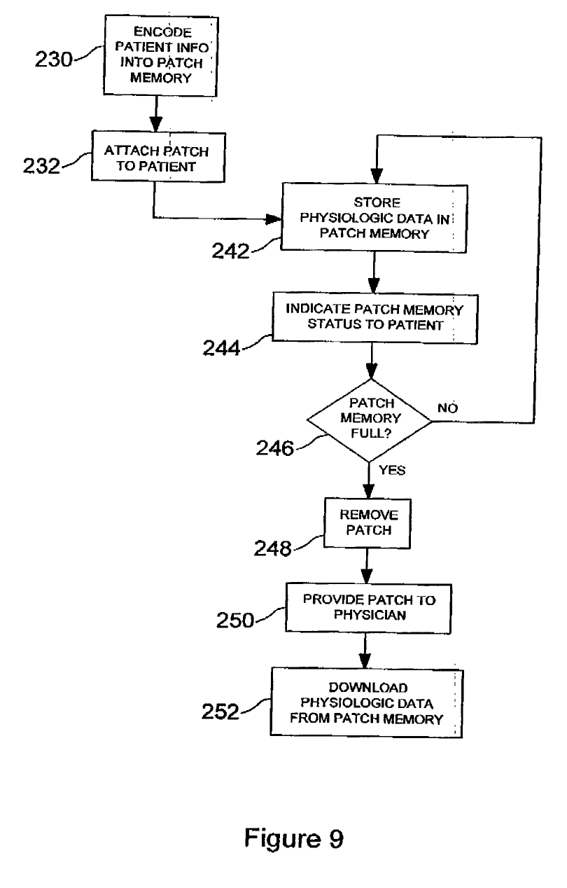

In general operation, and as further shown in FIG. 9, information concerning the patient is initially encoded 230 into memory 108 of peripheral monitor module 100 prior to use, typically at a physician's office. An input device or interface (not shown) is typically employed to facilitate encoding of patient information and device control parameters into memory 108. It is understood that this input device may be provided by replacing output device 212 with a suitable input/output interface. After encoding patient information into memory 108, peripheral monitor patch 100 is attached 232 to the patient 10. Peripheral monitor patch 100 includes an adhesive for affixing the patch 102 to the skin of the patient, or alternatively, to their clothes.

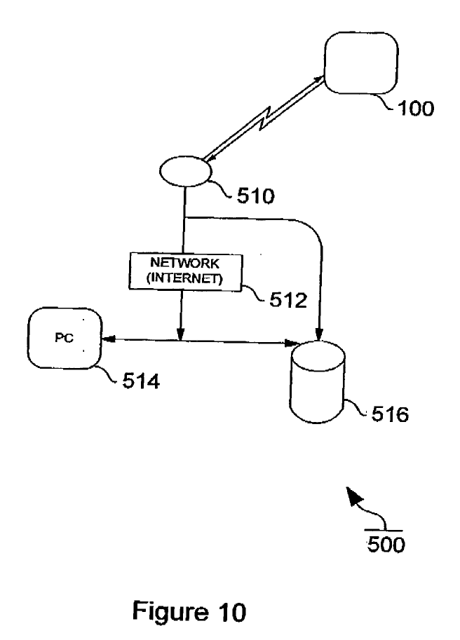

FIG. 10 represents remote communication system 500 wherein peripheral monitor 100 is in wireless data communication with interface 510 from which data collected by peripheral monitor 100 is transferred to network system 512. The data collected in network 512 is accessible to PC 514. Further, the data is archived/stored in data storage 516, which would be accessible also to PC 514. In this manner, the data collected by peripheral monitor 100 could be transferred to healthcare providers at a remote location using PC 514. The arrangement enables remote monitoring and chronic management of patients on a continuous, real-time basis.

Peripheral monitor patch 100 may include an indicator that provides a patient with an indication 244 of the status of memory 108. A visual or audible indicator may be provided on peripheral monitor patch 100 to provide a patient with a visual or audible indication of patch memory status and/or operating condition. If memory 108 is full or nearing zero capacity 246, peripheral monitor patch 202 may be removed from the skin by the patient 248 and subsequently provided to a physician 250 or other healthcare provider. It is contemplated that peripheral monitor patch 100 may be inserted into a standard mailing envelope and forwarded to the physician via regular mail.

Upon receiving peripheral monitor patch 100, personnel at a physician's office or healthcare clinic may download 252 physiologic and other data, such as medical device diagnostic information, stored in memory 108 of peripheral monitor patch 100. In one embodiment, peripheral monitor patch 100 is intended to be discarded after one use. Alternatively, the electronics module section of peripheral monitor patch 100 may be reused following an appropriate cleaning procedure and provided with a new adhesive tape or layer for subsequent or repeated use. It is noted that the power source 104, which may be a low profile battery, may be replaced to allow continued reuse of peripheral monitor module 100. Alternatively, it may be recharged for continued use.

The preceding specific embodiments are illustrative of the practice of the invention. It is to be understood, therefore, that other expedients known to those skilled in the art or disclosed herein may be employed without departing from the invention or the scope of the appended claims. It will be appreciated that a peripheral monitor patch apparatus and methodology according to the present invention may be implemented in several embodiments and is not limited to application in the devices described or referred to herein. The present invention is believed to find wide application to any form of peripheral monitor device that acquires physiologic data from a patient, which is subsequently or contemporaneously transmitted to a data storage or display device situated, remote from the patient. The present invention is believed to be particularly advantageous in those applications where physiologic data storage resources are required for extended periods of time.

The present invention is also not limited to specific data acquisition and communications techniques, such as those presented herein, but such functions may be directed using other like techniques. The present invention further includes within its scope methods of using a peripheral monitor patch as well as the structural particulars described herein-above.

In the claims, means plus function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. A non-tissue contacting electrode monitor system for sensing physiologic signals from a patient, the system comprising:
a flexible substrate with electronic sensors having data storage and a communication module incorporated thereon; and
at least one electrode positioned at a location spaced from intimate contact with a surface of the patient's body;
said substrate having an adhesive backing to enable wearability by the patient for sensing the physiologic signals via said at least one electrode for storage in said data storage and transmission, to a remote location from the patient, via said communication modules.

2. The system of claim 1 wherein said communication module is structured to transfer collected data to an external device.

3. The system of claim 1 wherein the monitor system includes a perceptible indicator to signal when said data storage is full.

4. The system of claim 3 wherein said flexible substrate is removable, when said data storage is fill, for downloading said stored data for a review by a physician.

5. The system of claim 1 wherein said communication module includes a telemetry system to transmit said physiological data to a remote location.
